(19) United States
(12) Patent Application Publication
(10) Pub. No.: US 2019/0076139 A1
(43) Pub. Date: Mar. 14, 2019

(54) EMBROIDERED TISSUE BUTTON

(71) Applicant: Arthrex, Inc., Naples, FL (US)

(72) Inventors: Peter J. Dreyfuss, Naples, FL (US); Thomas Dooney, Naples, FL (US); John Sodeika, Naples, FL (US); Orr Limpisvasti, Manhattan Beach, CA (US); Paul S. Shapiro, Bloomfield Hills, MI (US)

(21) Appl. No.: 15/704,473

(22) Filed: Sep. 14, 2017

(51) Int. Cl. A61B 17/04; A61B 17/06

(52) U.S. Cl. CPC A61B 17/0401; A61B 17/06166; A61B 2017/0429; A61B 2017/0414; A61B 2017/0404

(57) ABSTRACT

A surgical repair assembly includes a button including a body portion formed of embroidered yarn. The button includes at least one aperture. A suture strand engages the button to secure soft tissue to the bone, and the suture strand is received in the at least one aperture of the button.

### BACKGROUND OF THE INVENTION

A button is used in a rotator cuff surgical procedure to retain a tendon to a bone. A hole is drilled in the bone. A suture anchor is then inserted into the hole in the bone, and a suture strand attached to the suture anchor is used to secure the tendon to the bone. The button is located above the tendon, and the suture strand is tied to assist in securing the tendon to the bone.

The button can be made of stainless steel, titanium alloy, titanium, PEEK, PLLA or any material. As the button is solid, the tissue cannot incorporate into the button, allowing the button to shift over time.

### SUMMARY

A surgical repair assembly includes a button including a body portion formed of embroidered yarn. The button includes at least one aperture. A suture strand engages the button to secure soft tissue to the bone, and the suture strand is received in the at least one aperture of the button.

In one example, the suture anchor can be received in a hole in a bone, and the suture strand can engage the suture anchor. In one example, the body portion includes two apertures to receive the suture strand. Each of the two apertures can be oblong in shape. The tissue button can include a suture eyelet loop secured to the body portion of the tissue bottom. The suture eyelet loop can be integral with the body portion of the tissue button to define a single integral monolithic unitary component. The suture eyelet loop can also be secured to the body portion by glue or a knot. The yarn of the suture anchor can be polyester yarn.

A surgical repair assembly includes a suture anchor received in a hole in a bone. A tissue button includes a body portion formed of polyester embroidered yarn and a feature through which a suture strand is threaded. The suture strand engages the suture anchor and the tissue button to secure soft tissue to the bone. Soft tissue is secured between the tissue button and the bone.

In one example, the feature is two apertures, and the body portion includes the two apertures to receive the suture strand. Each of the two apertures can be oblong in shape. The feature can be a suture eyelet loop, and the tissue button includes the suture eyelet loop which is secured to the body portion of the tissue bottom. The suture eyelet loop is integral with the body portion of the tissue button to define a single integral monolithic unitary component. The feature can be a slit, and the tissue button includes the slit.

A method of performing a surgical repair includes inserting a suture anchor into a hole in a bone, passing the suture strand through soft tissue, passing the suture strand through a slit in a fixation button, pulling the suture strand through a knot pusher, pushing the fixation button towards the soft tissue, temporarily fixing the soft tissue against the bone with the fixation button, and securing the suture strand to secure the soft tissue to the bone after the step of temporarily fixing.

### BRIEF DESCRIPTION OF THE DRAWINGS

Other advantages of the present invention can be understood by reference to the following detailed description when considered in connection with the accompanying drawings wherein:

FIG. 1 schematically illustrates a surgically repaired shoulder including a suture anchor, an embroidered tissue button, bone, and a tendon of a rotator cuff;

FIG. 2 schematically illustrates an embroidered tissue button;

FIG. 3 schematically illustrates another surgically repaired shoulder including a suture anchor, another example embroidered tissue button, bone, and a tendon of a rotator cuff;

FIG. 4 schematically illustrates the another example embroidered tissue button;

FIG. 5 schematically illustrates a fixation button including a slot;

FIG. 6 schematically illustrates another surgical repair with a suture strand passing through the fixation button;

FIG. 7 schematically illustrates a knot pusher being moved towards the fixation button;

FIG. 8 schematically illustrates the knot pusher pressing on the fixation button;

FIG. 9 schematically illustrates the fixation button after the knot pusher is removed;

FIG. 10 schematically illustrates the surgical repair site after the suture strands are tied to secure the soft tissue to bone; and

FIG. 11 schematically illustrates the embroidered tissue button employed in an ankle syndesmosis.

### DETAILED DESCRIPTION OF THE PREFERRED EMBODIMENTS

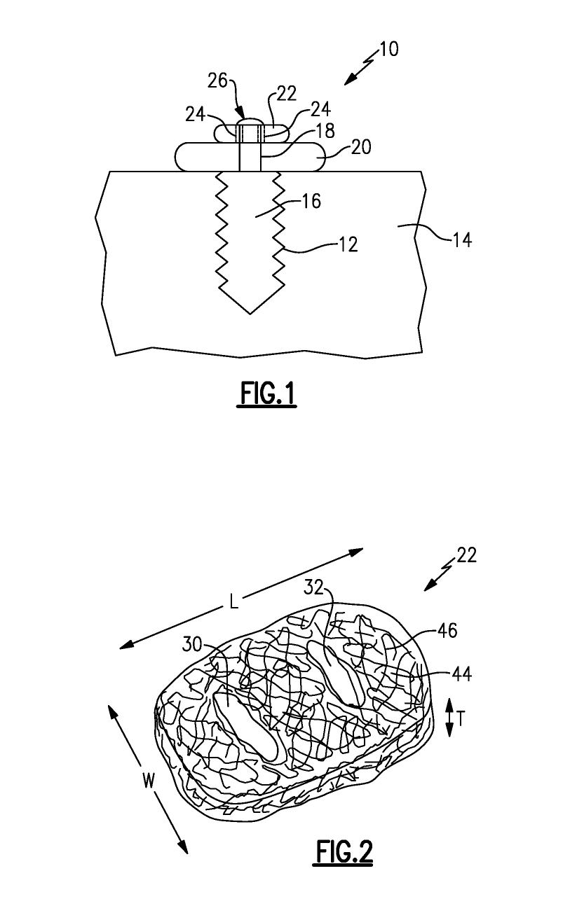

FIG. 1 schematically illustrates a surgically repaired shoulder. Four rotator cuff muscles are attached to a bone of the shoulder by rotator cuff tendons (only one tendon is shown schematically). If one of the rotator cuff tendons is torn, ripped, injured, damaged, etc., a surgical procedure can be employed to repair the tendon and/or reattach the tendon to the bone.

When repairing a rotator cuff tear, a hole 12 is drilled in the bone 14. A suture anchor 16 is then inserted in the hole 12 in the bone 14. A suture strand 18 is secured by the suture anchor 16 to the bone 14. In one example, the suture strand 18 is located around a rigid member in the suture anchor 16. In another example, the suture strand 18 is wedged between the suture anchor 16 and the bone 14.

The suture strand 18 is threaded through the tendon 20 (or other tissue, such as a ligament). An embroidered tissue button 22 is positioned above the tendon 20 and is employed to secure the tendon 20 to the bone 14 to repair the tear. The suture strand 18 passes through defined apertures 24 in the embroidered tissue button 22, and the suture strands 28 are tied in a knot 26 on top of the embroidered tissue button 22. After the suture strand 18 is tied, the embroidered tissue button 22 is fixed on top of the tendon 20. The tendon 20 is secured to the bone 14, and the tied suture strand 28 prevents the embroidered tissue button 22 from moving.

Although the embroidered tissue button 22 is disclosed and illustrated as being utilized in shoulder, it is to be understood that the embroidered tissue button 22 can be used in any surgical application. For example, the embroidered tissue button 28 can also be used in a procedure for acromioclavicular repair. Additionally, the embroidered tissue button 22 can also be used to secure a ligament or any soft tissue to bone or can be used in any another joint.

FIG. 2 illustrates an example of an embroidered tissue button 28. The embroidered tissue button 28 is manufactured by embroidery. The embroidered tissue button 28 can also be woven. In one example, the embroidered tissue button 28 is made of polyester yarn, and spaces 44 are defined between the yarn 46. The polyester yarn 46 can have any size or thickness. The embroidered tissue button 28 has a length L, a width W, and a thickness T. The largest dimension of the embroidered tissue button 28 is approximately 8 mm.

The embroidered tissue button 22 also includes two apertures 30 and 32. In one example, the embroidered tissue button 28 is round. In another example, the embroidered tissue button 28 is oblong. In one example, the apertures 30 and 32 are round. In another example, the apertures 30 and 32 are oblong. In one example, the apertures 30 and 32 together are centered relative to the embroidered tissue button 28.

The embroidered tissue button 28 has a tight mesh, and the embroidered tissue button 28 is less traumatic to the body. The spaces 44 in the embroidered tissue button 28 promote tissue ingrowth and capture between the yarn 46. During healing, the tissue can grow into the spaces 44 in the embroidered tissue button 22, securing the embroidered tissue button 22 in the body and preventing the embroidered tissue button 22 from floating in the body. As the embroidered tissue button 28 does not include any metal, there also is no x-ray artifact.

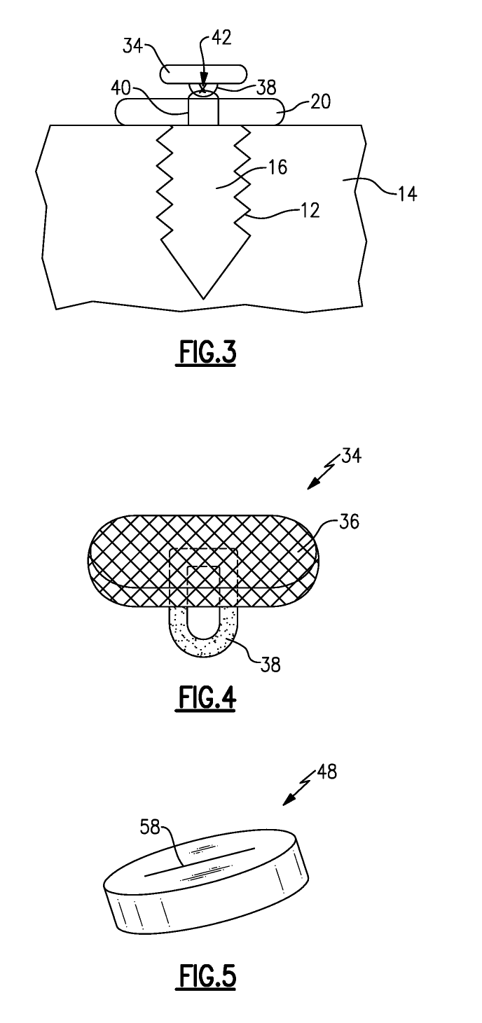

FIGS. 3 and 4 schematically illustrate another example of an embroidered tissue button 34. In this example, the embroidered tissue button 34 includes a body 36 formed of embroidered yarn as described above and a suture eyelet 38 that is attached to the body 36 of the embroidered tissue button 34. The suture eyelet 38 forms a suture loop. The suture eyelet 38 can be attached to the body 36 of the embroidered tissue button 34 by any means, such as threading, knotting, or gluing. Additionally, the suture eyelet 38 can be made integral with and be made of the same material as the body 36 to define a single integral monolithic unitary component.

In one example, the embroidered yarn is polyester yarn. The polyester yarn can have any size or thickness. The largest dimension of the embroidered tissue button 28 is approximately 8 mm.

In this embodiment, a suture strand 40 is threaded through the suture eyelet 38 to secure the tendon 20 to the bone 14. The embroidered tissue button 34 is located above the tendon 20. After threading the suture strand 40 through the tendon 20 and the suture eyelet 38, the suture strand 40 is tied in a knot 42 that is located between the tendon 20 and the embroidered tissue button 34. The embroidered tissue button 34 is fixed on top of tendon 20 to help secure the tendon 20 to the bone 14.

In FIG. 3, a space between the tendon 20 and the embroidered tissue button 34 is shown exaggerated in order to show the knot 42 more clearly. In use, the tendon 20 and the embroidered tissue button 34 contact, and the knot 42 is located therebetween.

FIG. 5 illustrates another embodiment of the invention. A fixation button 48 or washer is employed to temporarily fix soft tissue 56, such as a tendon or ligament, to a bone 52 prior to final fixation to confirm the soft tissue 56 fixated is in the ideal location.

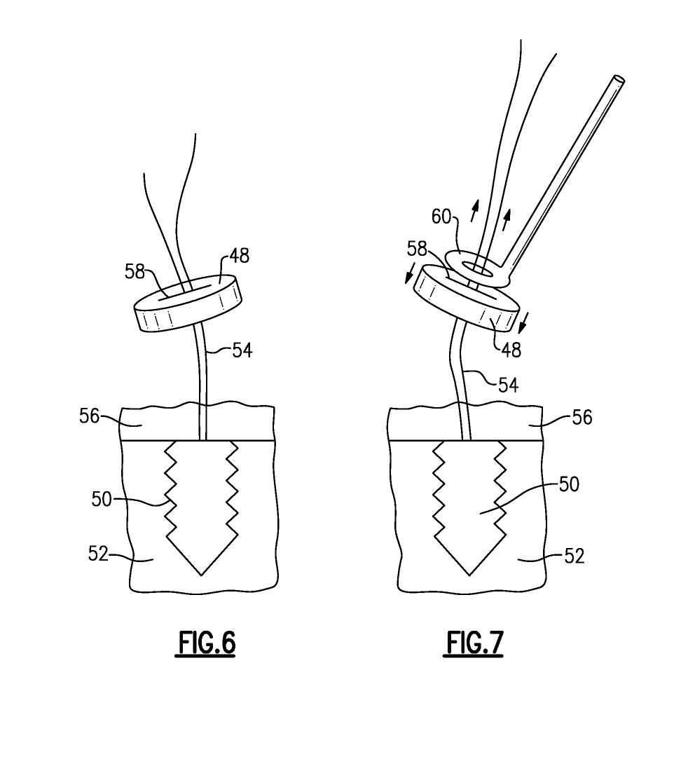

FIGS. 6 to 10 illustrate a method of using the fixation button 48. As shown in FIG. 6, a suture anchor 50 is inserted into the bone 52. A suture strand 54 (or suture tape) is secured relative to the bone 52 by the suture anchor 50. The suture strand 54 is passed through the soft tissue 56 and through a slot 58 in the fixation button 48. The slot 58 is thin and provides friction against the suture strand 54. The slot 58 holds the suture strand 54 in place, but allows the suture strand 54 to slide within the slot 58 if enough force is applied to the suture strand 54.

The fixation button 48 can be made out of any material. In one example, the fixation button 48 is made out of embroidery or an embroidered yarn. In one example, the fixation button 48 is embroidered from polyester yarn. The slot 58 can be formed by making a cut in the fixation button 48.

In FIG. 7, the suture strand 54 is pushed through an opening in a knot pusher 60, and the suture strands 54 are pulled while the fixation button 48 is pushed towards the soft tissue 56. In FIG. 8, the knot pusher 60 is pushed down on the fixation button 48 while the suture strands 54 are pulled relative to the fixation button 48.

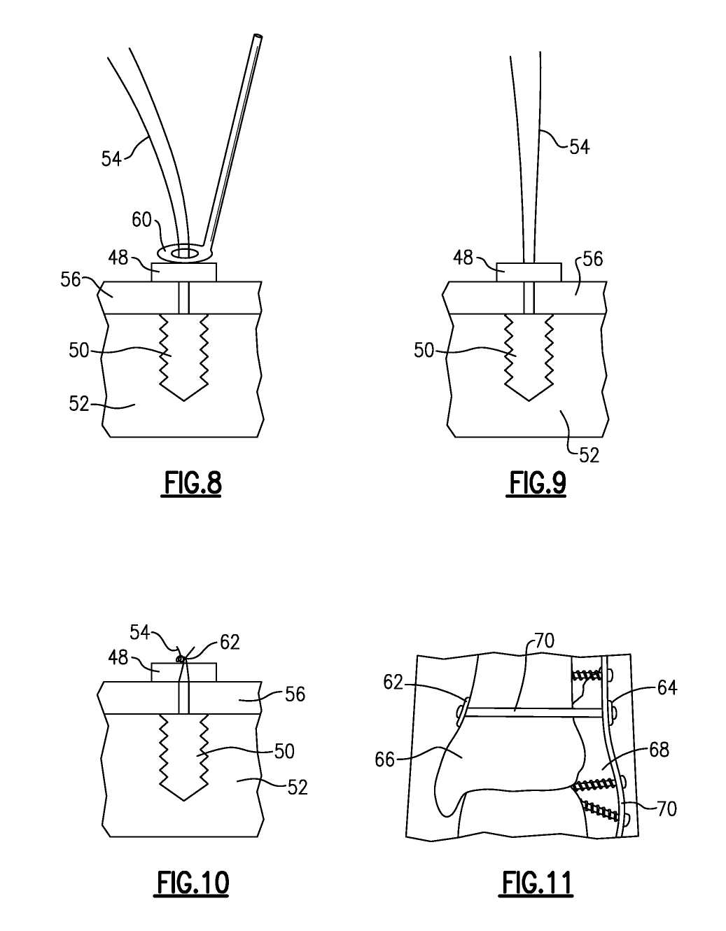

As shown in FIG. 9, the fixation button 48 temporarily fixes the soft tissue 56 against the bone 52. Finally, in FIG. 10, the suture strands 54 are tied in a knot 62 to secure soft tissue 56 to the bone 52. In another example, another suture anchor is employed to secure the soft tissue 56 to the bone 52.

The knot pusher 60 can also be employed with both the embroidered tissue buttons 22 or 34 described above.

FIG. 11 schematically illustrates the embroidered tissue button employed in an ankle syndesmosis to provide fixation of syndesmosis disruptions. In this example, a first embroidered button 62 and a second embroidered button 64 are employed. A suture 70 is tensioned and secured between the first embroidered button 62 and the second embroidered button 64, which are placed against a tibia 66 and a fibula 68, respectively, to stabilize the ankle. A plate 72 is located between the fibula 68 and the second embroidered button 64. The embroidered tissue button can be used in any application where a tissue button is employed. For example, the embroidered tissue button can also be used in a ligament reconstruction procedure, such as for a ligament in an ankle.

The foregoing description is only exemplary of the principles of the invention. Many modifications and variations of the present invention are possible in light of the above teachings. The preferred embodiments of this invention have been disclosed, however, so that one of ordinary skill in the art would recognize that certain modifications would come within the scope of this invention. It is, therefore, to be understood that within the scope of the appended claims, the invention may be practiced otherwise than as specifically described. For that reason the following claims should be studied to determine the true scope and content of this invention.

What is claimed is:

1. A surgical repair assembly comprising:
a button including a body portion formed of embroidered yarn, wherein the button includes at least one aperture; and
a suture strand engaging the button to secure tissue to a bone, wherein the suture strand is received in the at least one aperture of the button.

2. The surgical repair assembly as recited in claim 1 including a suture anchor received in a hole in the bone, wherein the suture strand engages the suture anchor.

3. The surgical repair assembly as recited in claim 1 wherein the tissue is soft tissue or bone.

4. The surgical repair assembly as recited in claim 1 wherein the yarn is polyester yarn.

5. The surgical repair assembly as recited in claim 1 wherein the tissue is the soft tissue that is secured between the button and the bone.

6. The surgical repair assembly as recited in claim 1 wherein the at least one aperture includes two apertures to receive the suture strand.

7. The surgical repair assembly as recited in claim 6 wherein each of the two apertures are oblong in shape.

8. The surgical repair assembly as recited in claim 1 wherein the button includes a suture eyelet loop secured to the body portion of the bottom.

9. The surgical repair assembly as recited in claim 8 wherein the suture eyelet loop is integral with the body portion of the button to define a single integral monolithic unitary component.

10. The surgical repair assembly as recited in claim 8 wherein the suture eyelet loop is secured to the body portion by glue or a knot.

11. The surgical repair assembly as recited in claim 1 wherein the button includes a slit.

12. A surgical repair assembly comprising:
a suture anchor received in a hole in a bone;
a tissue button including a body portion formed of polyester embroidered yarn and a feature through which a suture strand is threaded; and
the suture strand engaging the suture anchor and the tissue button to secure soft tissue to the bone, wherein soft tissue is secured between the tissue button and the bone.

13. The surgical repair assembly as recited in claim 12 wherein the feature is two apertures, and the body portion includes the two apertures to receive the suture strand.

14. The surgical repair assembly as recited in claim 12 wherein the feature is a suture eyelet loop, and the tissue button includes the suture eyelet loop which is secured to the body portion of the tissue bottom.

15. The surgical repair assembly as recited in claim 14 wherein the suture eyelet loop is integral with the body portion of the tissue button to define a single integral monolithic unitary component.

16. The surgical repair assembly as recited in claim 12 wherein the feature is a slit, and the tissue button includes the slit.

17. A method of performing a surgical repair, the method comprising the steps of:
inserting a suture anchor into a hole in a bone;
passing the suture strand through soft tissue;
passing the suture strand through a slit in a fixation button;
pulling the suture strand through a knot pusher;
pushing the fixation button towards the soft tissue;
temporarily fixing the soft tissue against the bone with the fixation button; and
securing the suture strand to secure the soft tissue to the bone after the step of temporarily fixing.

18. The method as recited in claim 17 wherein the fixation button is formed of an embroidered yarn.

19. The method as recited in claim 18 wherein the yarn is polyester yarn.

20. The method as recited in claim 19 including the step of providing friction between the suture strand and the fixation button.
